(19) United States
(12) Patent Application Publication
(10) Pub. No.: US 2014/0081090 A1
(43) Pub. Date: Mar. 20, 2014

(54) PROVISION OF ATYPICAL BRAIN ACTIVITY ALERTS

(71) Applicant: Affectiva, Inc., Waltham, MA (US)

(72) Inventors: Rosalind Wright Picard, Newtonville, MA (US); Daniel Bender, Cambridge, MA (US); Walter Gillett, Lexington, MA (US)

(73) Assignee: Affectiva, Inc., Waltham, MA (US)

(21) Appl. No.: 14/079,723

(22) Filed: Nov. 14, 2013

Related U.S. Application Data
(63) Continuation-in-part of application No. 13/153,745, filed on Jun. 6, 2011.
Application No. 13/153,745 is a continuation-in-part of application No. 13/624,894, filed on Sep. 22, 2012.
Application No. 13/624,894 is a continuation-in-part of application No. 13/624,898, filed on Sep. 22, 2012.
(60) Provisional application No. 61/726,553, filed on Nov. 14, 2012.
(60) Provisional application No. 61/747,651, filed on Dec. 31, 2012.
(60) Provisional application No. 61/747,810, filed on Dec. 31, 2012.
(60) Provisional application No. 61/793,761, filed on Mar. 15, 2013.
(60) Provisional application No. 61/790,461, filed on Mar. 15, 2013.
(60) Provisional application No. 61/789,038, filed on Mar. 15, 2013.
(60) Provisional application No. 61/798,731, filed on Mar. 15, 2013.
(60) Provisional application No. 61/844,478, filed on Jul. 10, 2013.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.
(60) Provisional application No. 61/538,218, filed on Sep. 23, 2011.
(60) Provisional application No. 61/538,218, filed on Sep. 23, 2011.

(51) Int. Cl. A61B 5/00; A61B 5/11; A61B 5/02; A61B 5/16; A61B 5/18; A61B 5/053; A61B 5/01

(52) U.S. Cl. CPC A61B 5/4064; A61B 5/0531; A61B 5/1112; A61B 5/01; A61B 5/7282; A61B 5/6898; A61B 5/4094; A61B 5/4848; A61B 5/165; A61B 5/4806; A61B 5/18; A61B 5/168; A61B 5/4884; A61B 5/02028; A61B 5/746; A61B 5/7267; A61B 5/486; A61B 5/0022; A61B 5/681; A61B 5/167
USPC 600/301; 600/300; 600/547; 600/595; 600/549

(57) ABSTRACT

Computer-implemented techniques are disclosed for analyzing physiology data captured by biosensors and for providing alerts based on the biosensor data. The biosensor data is captured non-invasively and is analyzed to identify atypical brain activity that can result from illness, injury, disease, and seizures. The biosensor data can include electrodermal activity, skin temperature, and accelerometer data. The data is sent to a web service for analysis, and to determine whether an alert should be generated. The alerts, based on the biosensor data captured from the individual, are received from the web service. An output related to physiology is rendered based on an alert being received from the web service.

### RELATED APPLICATIONS

This application claims the benefit of U.S. provisional patent applications “Provision of Atypical Brain Activity Alerts” Ser. No. 61/726,553, filed Nov. 14, 2012, “Optimizing Media Based on Mental State Analysis” Ser. No. 61/747,651, filed Dec. 31, 2012, “Collection of Affect Data from Multiple Mobile Devices” Ser. No. 61/747,810, filed Dec. 31, 2012, “Mental State Analysis Using Heart Rate Collection Based on Video Imagery” Ser. No. 61/793,761, filed Mar. 15, 2013, “Mental State Data Tagging for Data Collected from Multiple Sources” Ser. No. 61/790,461, filed Mar. 15, 2013, “Mental State Analysis Using Blink Rate” Ser. No. 61/789,038, filed Mar. 15, 2013, “Mental State Well Being Monitoring” Ser. No. 61/798,731, filed Mar. 15, 2013, and “Personal Emotional Profile Generation” Ser. No. 61/844,478, filed Jul. 10, 2013. This application is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Data Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011. This application is also a continuation-in-part of US patent application “Analysis of Physiology Based on Electrodermal Activity” Ser. No. 13/624,894, filed Sep. 22, 2012, which claims the benefit of U.S. provisional patent application “Analysis of Physiology Based on Electrodermal Activity” Ser. No. 61/538,218, filed Sep. 23, 2011. This application is also a continuation-in-part of US patent application “Clinical Analysis Using Electrodermal Activity” Ser. No. 13/624,898, filed Sep. 22, 2012, which claims the benefit of U.S. provisional patent application “Analysis of Physiology Based on Electrodermal Activity” Ser. No. 61/538,218, filed Sep. 23, 2011. The foregoing applications are each hereby incorporated by reference in their entirety.

### FIELD OF ART

This application relates generally to the analysis of physiology and more particularly to the provision of atypical brain activity alerts.

### BACKGROUND

The medical electronics market has grown significantly over recent decades, providing substantial improvements in healthcare to providers and recipients. The large sector of electronic medical equipment encompasses a wide range of devices for applications that include remote patient monitoring, wireless healthcare and services, consumer medical electronic devices, invasive and non-invasive procedures such as diagnostics, imaging, treatment, implants, so-called “pharma-technologies” (e.g. pacemakers and drug pumps), and so on. One of the largest segments of the medical electronics market is consumer medical electronic devices. Further, the scope and function of medical electronic devices are now being widely adopted to meet a variety of medical demands. Such demands include cost-effective solutions to meet the variety of changing medical needs of various demographics, including the world's aging population. Low-cost electronic components are also highly desirable to meet the medical needs of remote, poor, or rural populations requiring healthcare. These electronic components are often included in disposable items that now may be distributed widely to meet healthcare needs due to the low cost of the electronic components. Further, rates of disease and chronic conditions have remained static or are even increasing, in part due to better diagnostics supported by medical electronic devices. And, new diagnostic techniques require additional applications of medical electronic devices. Such increased demand for the devices continues to drive improvements such as reduced size, increased device and system feature sets and capabilities, better medical data communications standards, better data encryption to protect medical data, and improved power management to ensure the longevity and reliability of the medical devices.

The evaluation of an individual's physiological condition, as well as the evaluation of other parameters, can be extremely advantageous in forming a determination of the individual's level of overall health and activity. The types of physiological information that may be gathered from an individual comprise a variety of parameters, including electrodermal activity (EDA)—also known as skin conductance (SC) or galvanic skin response (GSR). Such physiological information may further include skin temperature, heart rate, heart rate variability, and various other data measures pertaining to the status of a human. The tracking of body movement may also be beneficial, and may be collected through readings of devices such as accelerometers. All of the data readings, as well as other information about the individual, may be collected to evaluate the overall health of the individual, to diagnose numerous health problems, and/or to track physical or exercise activity.

A biosensor attached to the human body may be used to provide physiological readings and other pertinent information. Previous implementations of most biosensors have been cumbersome, obtrusive, and difficult to use. The presence of such an obtrusive biosensor often impacts the user's readings simply because the wearer is cognizant of the attached biosensor. Electrodermal activity varies with multiple factors, including the moisture level of the skin. The moisture level of the skin is a function of the body's sweat glands, which are controlled by the sympathetic nervous system. Therefore, skin conductance provides an indication of the psychological or physiological state of a person. A long history of electrodermal activity research, which dates back to the 1800s, has mostly focused on the spontaneous fluctuations or reactions of electrodermal activity to stimuli.

### SUMMARY

Biosensor data is captured and analyzed to determine a physiological condition and provide an alert associated with the physiological condition. A computer-implemented method for physiology analysis is disclosed comprising: capturing biosensor data from a person; sending the biosensor data to a mobile device; providing the biosensor data to a cloud computing environment; processing the biosensor data to identify atypical brain activity; and providing an alert based on the atypical brain activity that was detected. Training can occur for the computer algorithm for the processing the biosensor data to identify the atypical brain activity. The training can include information on the alerts and/or cancelling of the alerts. Quantitative self-report information can be used to aid in tailoring alerts.

In embodiments, a computer-implemented method for physiology analysis comprises: receiving captured biosensor data from a person into a web-server computer system; analyzing the biosensor data from the person which was captured to identify atypical brain activity; and providing an alert based on the atypical brain activity that was detected. In some embodiments, a computer-implemented method for analyzing physiology comprises: receiving an alert based on: biosensor data which was captured from a person; and an analysis which was performed on a web service on the biosensor data wherein the analysis identified atypical brain activity.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram describing providing alerts based on biosensor data.

FIG. 2 is a diagram showing a system for providing alerts.

FIG. 3 is a diagram showing a system for gathering alerts.

FIG. 4 is a diagram showing a biosensor and a charging mat.

FIG. 5 is a diagram representing physiological analysis.

FIG. 6 is a flow diagram for providing alerts.

FIG. 7 is a flow diagram for receiving alerts.

FIG. 8 is a system diagram for physiology analysis.

### DETAILED DESCRIPTION

The present disclosure provides a description of various methods, apparatus, and systems for analysis of physiology along with providing associated alerts. Various types of biosensor data can be collected including electrodermal activity, skin temperature, and accelerometer data. Electrodermal activity reflects autonomic nervous system activity, and thus provides insight into an individual's physical or mental state. In particular, some electrodermal activity may exhibit a signature or characteristic that is associated with a physiological condition. Many such conditions are described herein, and still others will be appreciated, including pain, anxiety, panic attacks, epileptic seizures, sleep disorders, heart attacks, and the like.

By gathering data from a group of people in known contexts or experiencing known physiological conditions, it becomes possible to extract signatures in electrodermal activity data by noting correlations between the data profiles of each person in the group of people. Knowing these correlations allows for searching of signatures in the electrodermal activity data of an individual. When the electrodermal signatures are found in the electrodermal activity data of the individual, the physiological condition of the individual at the time the signature appeared in the electrodermal activity data may be determined or inferred. Specifically, the physiological condition of the individual at that time may be matched with a known physiological condition associated with a certain signature in the collected data from the plurality of other people.

In embodiments, the computation required to carry out the correlating, determining, inferring, and so on may occur on a client, a server, in part on a client and in part on a server, or the like. Although a variety of applications of the methods, apparatus, and systems described herein will become apparent in light of this disclosure, some applications include market research (e.g., determining visceral reactions to an advertisement or product presentation), clinical trials (e.g., determining how well or poorly an individual is reacting to a treatment or how well or poorly an individual is complying with a treatment protocol), and so forth.

Throughout this disclosure, the terms “identify” and “infer” may be used interchangeably to mean to deduce information derived from evidence rather than simply from explicit statements. Inference can include inference using probabilistic models; for example, the inference may be a 0.6 probability of a condition being present in current data. Throughout this disclosure, the phrases “other individuals” and “plurality of other people” may be used interchangeably. Throughout this disclosure, the words “signature” and “characteristic” may be used interchangeably. Although the term “biomarker” as used in the art may generally refer to a substance used as an indicator of a biological state (e.g., percent oxygenation of blood), a biomarker, as described herein, may include a physical, objective measurement of an individual; a measurement of an ability of an individual to conduct an electric signal; changes over time in that electrical signal or the ability to conduct the signal; simultaneous differences in the signal or in the ability to conduct the signal at various appendages of an individual; or the like. Throughout this disclosure, the term physiology may include psychophysiology. Likewise, when a physiological condition is referenced, the reference may include a psycho-physiological condition, unless otherwise stated or clear from the context. It should be understood that individuals may include humans.

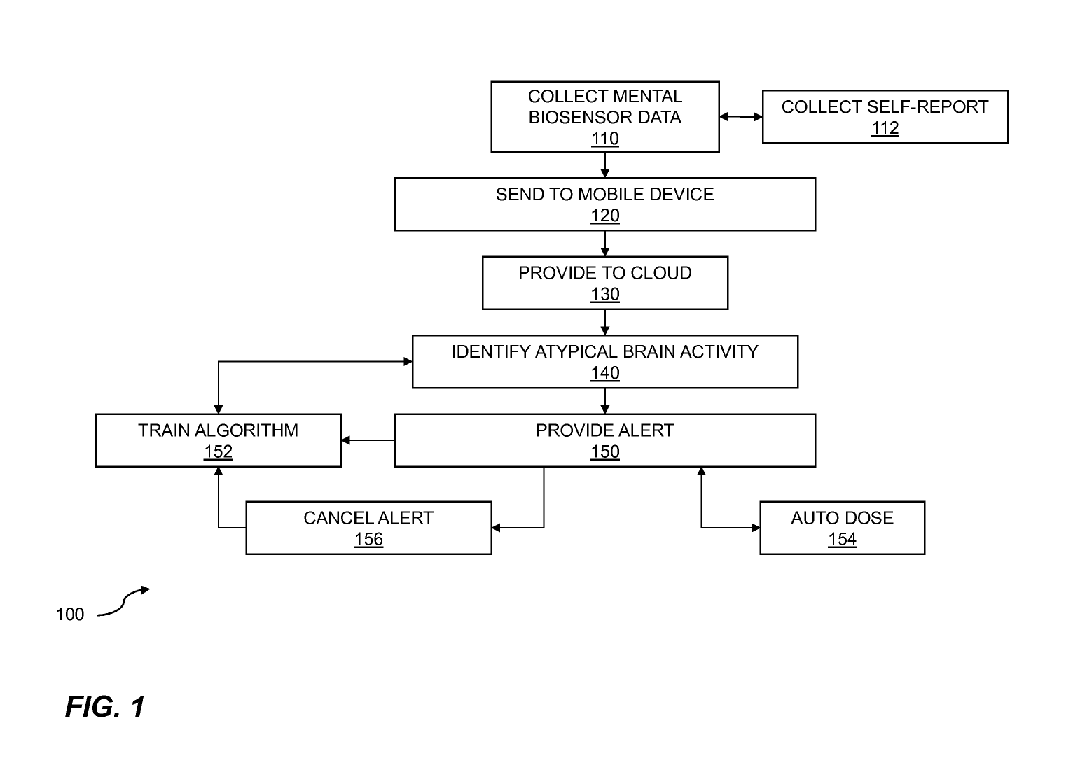

FIG. 1 is a flow diagram describing the providing of alerts based on biosensor data. The flow 100 includes capturing biosensor data from a person 110; sending the biosensor data to a mobile device 120; providing the biosensor data to a cloud computing environment 130; processing the biosensor data to identify atypical brain activity 140; and providing an alert based on the atypical brain activity that was detected 150. The biosensor data may include one or more of electrodermal activity, accelerometer data, and skin temperature. In some embodiments, ECG, EEG, PPG, or other physiological data could be collected. The processing may be performed in the cloud-computing environment. The mobile device may be incorporated as part of the biosensor. The atypical brain activity may comprise a SUDEP risk, a epileptiform seizure, a drug reaction, stress, depression, compulsive behavior, ictal brain activity, inter-ictal brain activity, tonic-clonic seizures, partial complex seizures, pain, anxiety, panic attacks, epileptic seizures, sleep disorders, respiratory sleep problems, heart attack, depression, stress, reaction to medication, bipolar attack, distracted driving, concussion, stroke, autistic reaction, ADHD behavior, boredom, being startled, being in a mood, eczema outbreak, fragile X syndrome reaction, obsessive-compulsive disorder, phobia, post-traumatic stress disorder, and social anxiety disorder as some examples. In some embodiments, other events such as exercise data, blood sugar changes, insulin reminders, repetitive activity warnings, the need for a rest or break period, the need for deep breathing, a reminder for calling a sponsor, or the like could be provided. The method may further comprise training 152 a computer algorithm for the processing the biosensor data to identify atypical brain activity. The training may be accomplished using a support vector machine where the training identifies an atypical brain activity for the person. The identification may be based on information stored in the cloud-computing environment for a group of people as well as information on the person. The method may further comprise generating a confidence score for the alert for the atypical brain activity. The method may further comprise cancelling an alert 156. The cancelling of an alert may be used to further train the computer algorithm to identify the atypical brain activity. The training the computer algorithm may be based on feedback after the alert. The training can be personalized for an individual and can aid in identifying if a person has a high or low risk for SUDEP or other phenomenon. The method may further comprise collecting quantitative self-report information 112 from the person. The self-report information can be collected ahead of time and the alert threshold and system can be tailored for the individual. Analysis can increase sensitivity to various factors to alert for including depression, anxiety, and others. The alert may be sent to one or more of the person, a caregiver, a relative, and a physician. The alert can also be used to log data and this log can be shared, if desired, with other appropriate people. These alerts could be incorporated into an overall personal health record, well-being status indicator, or the like. The method may further comprise auto-dosing 154 the person based on the atypical brain activity that was identified. The dosing can include calculating of an injection, drug, treatment, electrical stimuli, mechanical stimuli, or the like. The processing the biosensor data to identify atypical brain activity may be based on a threshold value.

Embodiments of the disclosed concepts include systems and methods to characterize seizures. This characterization can include a combination of on-board-device detection and cloud-based characterization. A display can provide data to a patient and a doctor and can further be used to infer treatment efficacy as well as recommend action based on the characterization. Personalized feedback on a seizure can be provided such as timing, morphology, autonomic disruption, and other elements. These various elements can be tracked for each seizure to determine progress. Activities of the person can also be tracked, such as sleep, stress, and other activities to help health care professions and patients determine any triggers for the atypical brain activity. In various cases, sensors can be worn on limbs on both sides of the body—either both wrists or both legs. Sensors can measure three-axis accelerometer data, electrodermal activity, and skin temperature, as well as other parameters possibly including photoplethysmogram information, heart rate, and heart rate variability. This biosensor data may be measured during the seizure as well as throughout non-seizure (regular) activities. Some measurements to evaluate may include asymmetry of electrodermal activity, asymmetry of movement, and asymmetry of changes in skin temperature during the seizure, as well as numerous other measurements. Analysis of the electrodermal activity may include evaluation of the energy or amplitude in one or more frequency bins and by filtering followed by amplitude evaluation of the median, average, or peak values over adjacent windows in time. Machine learning may be used to train an algorithm to learn the morphology of the peak sections involved in the seizure. Multiple features, including examination of rise time, sustained time, fall time and a build vector that characterizes this morphology, may characterize each seizure. Distances between vectors can be computed to determine similarity between seizures. Similar seizures may be grouped within an individual and across individuals using clustering algorithms.

Similar features may be computed for non-seizure data and non-seizure activity, and these features can also be grouped. False alarms may be used to help evaluate seizure-detection algorithms. For example, if false atypical brain activity is detected while playing a computer game, a “false alarm” button may be pressed. That data may be used to train the machine-learning algorithm. The analysis may group the false alarms versus the true seizures as two different categories, and may include retraining the algorithm to improve its discrimination. Seizures can be characterized in many ways: for example, one seizure may have highly asymmetric accelerometer activation measured from the two lower calf regions; electrodermal activity that slowly climbs, then rapidly climbs, then stays high for 20 minutes before beginning decay; and a drop in skin temperature on one side of the body only. A patient and his/her caregivers can get a read-out characterizing each seizure's physiological morphology, along with time of day and location information for the person when that morphological form occurred, since time of day may be important for chronotherapy, and GPS location may be important for bringing aid. A user may have an interface to customize with a desired sensitivity and specificity of the algorithm, the nature of the alert, and so on. A patient and his/her caregivers can also get read-outs characterizing the following patterns that preceded the seizure: overall SNS activation for the day before, overall physical activity for the day before, overall sleep patterns for the night before, and a score of sleep regularity and sleep quality for the week before. Sleep regularity and sleep quality may be detected by other means as well. Seizure feature patterns may be used to train a machine-learning algorithm to discriminate a seizure from non-seizure activities. The system may begin with using a machine learning algorithm trained on a group of patients with a similar suspected or confirmed diagnosis and applying this learning to a new patient's seizures. False positives may be identified when a patient presses a button on the device during the seizure. A patient's seizure may be compared to his/her previous seizures. After collecting biosensor data on multiple seizures from a patient, the results from this patient may be clustered with others who have had seizures with similar morphology. Similar treatment may then be evaluated. This method or a similar one could be used with a broader population and could be used for monitoring of health, employee exercise, employees being sedentary, carpal tunnel related motions, at risk behavior, and other types of monitoring. Various steps in the flow 100 may be changed in order, repeated, omitted, or the like without departing from the disclosed concepts. Various embodiments of the flow 100 may be included in a computer program product embodied in a non-transitory computer readable medium that includes code executable by one or more processors.

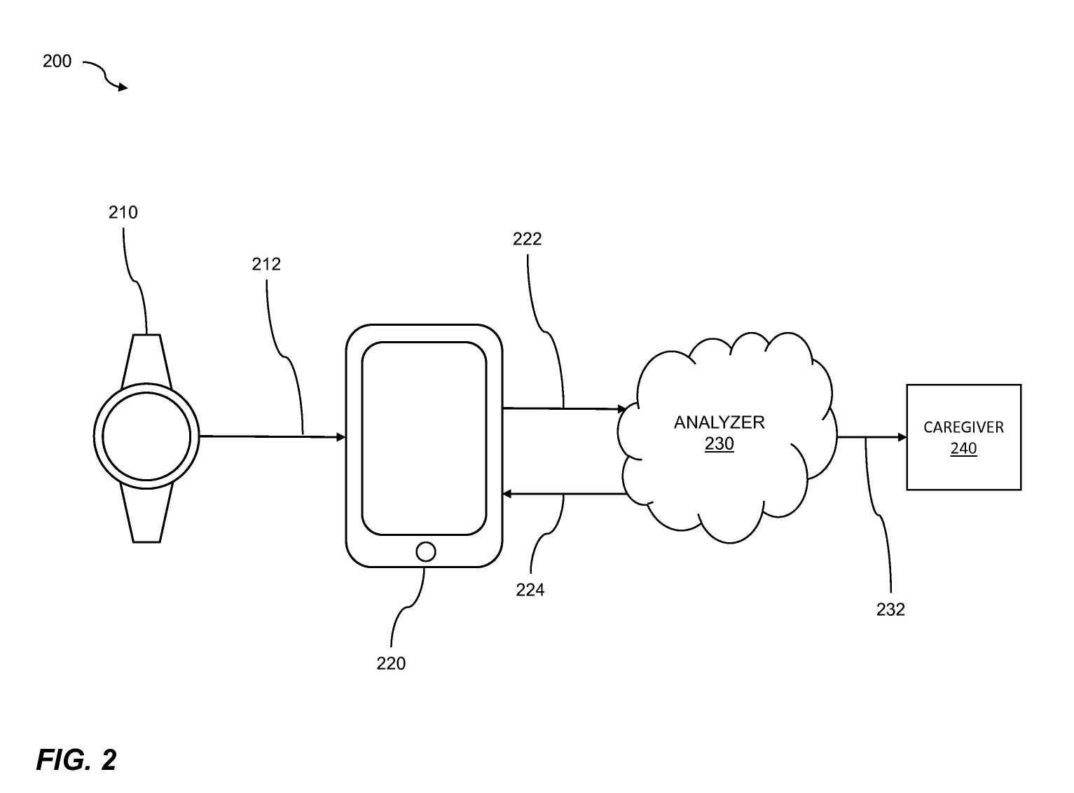

FIG. 2 is a diagram showing a system for providing alerts. The diagram 200 shows a system for implementing a computer-implemented method for physiology analysis. Physiological data may be captured and analyzed for atypical brain activity. The method may include using one or more biosensors for capturing biosensor data from a person, and a system for data analysis of the biosensor data. A biosensor 210 may be used for physiology analysis of a person. The one or more biosensors may be worn by the person from whom data is to be captured, placed on the person, be adjacent to the person, and so on. The biosensor data may include electrodermal activity, accelerometer data, skin temperature, heart rate, heart rate variability, and so on. The one or more biosensors may be worn on a wrist or other portion of a body. The biosensor 210 may send biosensor data to a mobile device 220. The data may be sent from the biosensor to the mobile device via a wired connection or a wireless connection. The data may be streamed 212. In other embodiments, the data may be sent in packets, sent in bursts, or sent via any other appropriate communication technique. The mobile device may be any suitable device including a smart watch, a cell phone, a PDA, a tablet, a laptop computer, and so on. The mobile device, in addition to receiving the biosensor data, may log the data, filter the data, process the data, and so on. In embodiments, the mobile device may detect suspicious activity contained in the biosensor data, and may relay the biosensor data to a cloud service for analysis.

The mobile device 220 may provide further analysis and/or may provide the biosensor data to an analyzer 230 including a web service in a cloud-computing environment. The biosensor data may be provided to the cloud-computing environment via streaming 222 or via any other appropriate data communication technique such as packets, bursts, and so on. The cloud computing system 230 may analyze the biosensor data to identify atypical brain activity. Atypical brain activity may include a SUDEP risk, an epileptiform seizure, a drug reaction, stress, depression, compulsive behavior, ictal brain activity, inter-ictal brain activity, tonic-clonic seizures, partial complex seizures, pain, anxiety, panic attacks, epileptic seizures, sleep disorder, respiratory sleep problems, heart attack, depression, stress, reaction to medication, bipolar attack, distracted driving, concussion, stroke, autistic reaction, ADHD behavior, boredom, being startled, being in a mood, eczema outbreak, fragile X syndrome reaction, obsessive-compulsive disorder, phobia, post-traumatic stress disorder, and social anxiety disorder, among others. Biosensor data from a plurality of people may be received and analyzed. For example, SUDEP analysis may include receiving biosensor data from a plurality of people sited at various locations. The processing of the biosensor data may be performed to identify atypical brain activity, including SUDEP risk. In the event that a high level of SUDEP risk is determined by the analysis, an alert or plurality of alerts may be provided based on the atypical brain activity that was detected. The alert or alerts may be triggered 224 on the mobile device. The alert or alerts may be visual alerts, audio alerts, and a combination of visual and audio alerts. When atypical brain activity has been identified, an alert or plurality of alerts may be sent to a caregiver 240 or another person or computer system. The other person or persons to whom an alert or alerts may be sent include, in embodiments, a relative, a physician, another healthcare professional, and so on. A computer system that may receive an alert may include an analysis system, a data logging system, a messaging system, and so on. The messages may be sent 232 in any appropriate format including SMS text, voice messages, prerecorded messages, and so on. In embodiments, an alert can be replaced with an intervention, a logging of information to a database, a titration of dosage, gathering of metadata about the atypical brain activity, and so on. Data could also be gathered from a GPS location capability, a request for further information could be provided, or similar further collection.

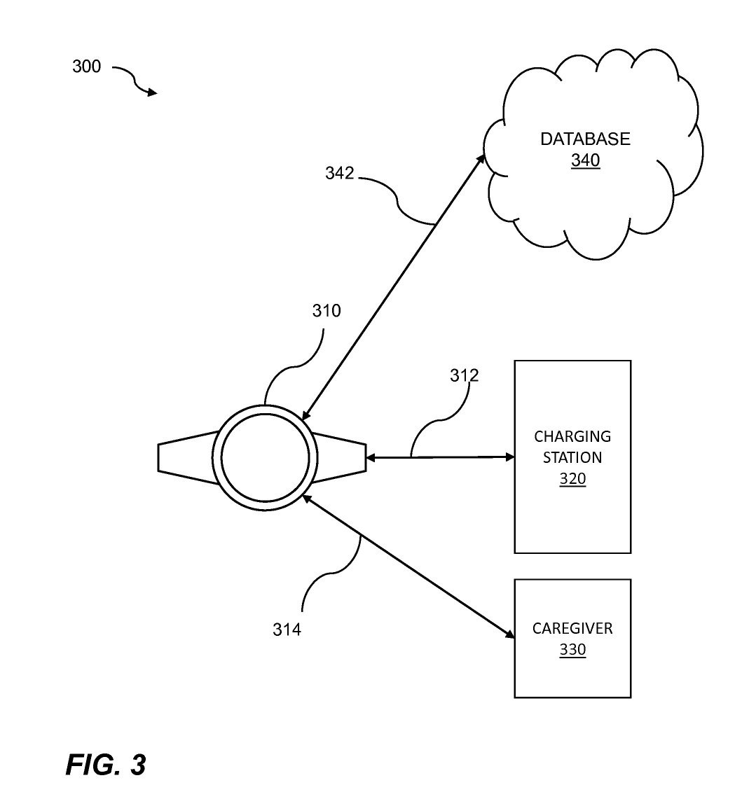

FIG. 3 is a diagram showing a system for gathering alerts. A biosensor 310 or a plurality of biosensors may be used for the collection of physiological data from a person. The data captured from the biosensor or plurality of biosensors may include electrodermal activity (skin conductance), accelerometer data, heart rate, heart rate variability, skin temperature, and so on. In embodiments, a biosensor 310 may be worn on a person's wrist. In other embodiments, a biosensor may be worn at other locations on the person's body, placed on the person, placed adjacent to the person, and so on. The biosensor may take an appearance similar to, or be incorporated as part of a wristwatch. In other embodiments, the biosensor may take the form of a piece of jewelry such as a pendant or brooch, a band, a tag, a patch, and so on. In embodiments, the mobile device may be incorporated as part of a biosensor. The mobile device may perform some processing on the biosensor data, or may provide the data to another processing environment such as a cloud-computing environment. The mobile device may include a smart watch, a cell phone, a PDA, a tablet, and so on.

A charging mat 320 may inductively charge the biosensor 310 when it is nearby or in contact with the charging mat. The charging mat may upload 312 biosensor data from the biosensor or biosensors. The upload of data may be based on wireless communication techniques, wired communication techniques, and so on. The biosensor data may be streamed, packetized, sent in bursts of data, and so on. For example, wireless-based communications may include Wi-Fi and/or Bluetooth, minimum-transmission-energy approaches, and so on. Wired communications may be based on direct connection to the biosensor using any appropriate connector, contact, pin, and so on. The charging may be based on quick charging, trickle charging, inductive charging, and so on.

As stated above, a mobile device may be incorporated as part of biosensor 310. The mobile device may perform some processing of the biosensor data and/or provide the biosensor data to another processing environment such as a cloud-computing environment. The processing may identify atypical brain activity in the person wearing the biosensor. Atypical brain activity may include SUDEP risk. In the event that the processing identifies atypical brain activity, an alert may be sent 314 to a caregiver 330. The alert may be a visual alert, an audio alert, and a combination of visual and audio alerts. The alert or a plurality of alerts may include an SMS message, a prerecorded voice call, and so on. The alert may be sent to one or more of the person using the biosensor, a caregiver, a physician, a healthcare professional, a relative and so on. The biosensor 310 may communicate alerts for atypical brain activity either directly or through a cloud-based computer system. The alerts may be communicated to a computer, mobile device, or other device to which the caregiver has access.

Data that is collected by the charging station and data hub 320 may be uploaded to a database 340. The upload may be a direct upload 342 from the biosensor 310 or may pass through the charging station 320. The database may be a cloud-based database, a web database, a research database, a local database, and so on. The upload of data from the data hub may be performed on a schedule. The schedule may be based on the timing of charging the biosensor, or may be based on a data transfer schedule appropriate for data analysis, research, or another appropriate activity. The biosensor data may be collected in a database for a specific research purpose, such as epilepsy research. Biosensor data may be collected from multiple biosensor users where the users may be at different locations. The results of the analysis of the data in the research database that has been collected may be used for general research purposes, specific research purposes, to improve detection of atypical brain activity, to improve algorithms for detection, and so on.

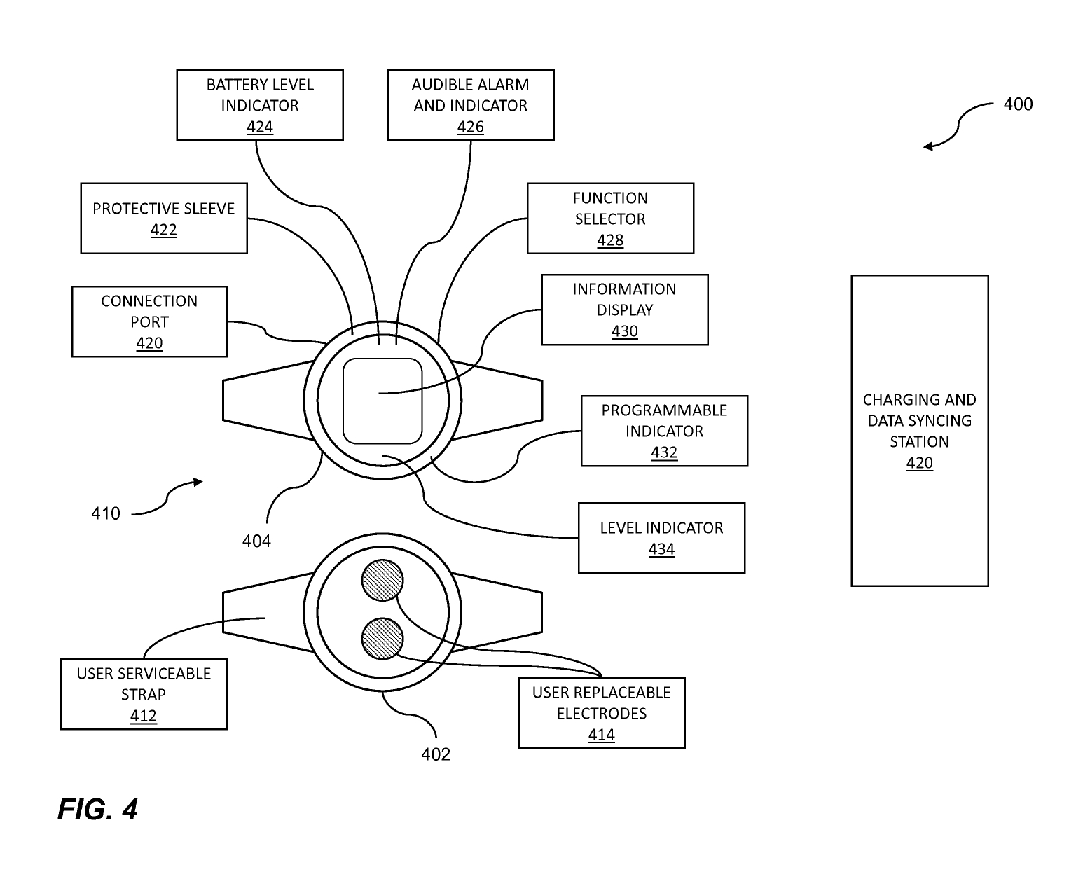

FIG. 4 is a diagram showing a biosensor 410 and a charging mat 420. The system 400 may be used for capturing biosensor data from a person. A mobile device may be incorporated as part of a biosensor and may add features and functions that aid in usability of the biosensor. The mobile device may be a smart watch, a cell phone, a tablet, a laptop, and so on. In embodiments, the biosensor may take the form of a watch. The watch-form biosensor may be worn on the wrist of the person. The watch may have a user-serviceable strap 412. The strap may be shortened, replaced, serviced, substituted, and so on to improve comfort, to extend the useful life of the biosensor, and so on. The strap may be a silicone strap. The back of the biosensor watch 402 may include user-replaceable electrodes 414. The electrodes may make a connection to the person using the biosensor. The electrodes may be used to collect biosensor data including electrodermal activity, accelerometer data, heart rate, heart rate variability, skin temperature, and so on. In embodiments, the user replaceable electrodes are silver/silver chloride electrodes. Any electrodes appropriate to the purposes of biosensing may be used.

The biosensor 410 collects biosensor data from a person. The biosensor may perform some processing of the biosensor data, the results of which may be used to determine when to send an alert to the person due to atypical brain activity. An example of a watch-form biosensor 404 that may incorporate a mobile device is shown. The biosensor watch may include functions and features that aid in capturing biosensor data. Data may be sent to or captured from the biosensor using a connection port 420. The connection port may be used for charging the biosensor, programming the biosensor, capturing data from the biosensor, and so on. In embodiments, the connection port is a micro-USB port. The biosensor may include a protective sleeve 422. The protective sleeve may be used to protect the biosensor from the environment, may be used to protect the user of the biosensor, and so on. In embodiments, the protective sleeve is a silicone protective sleeve. The biosensor may include a variety of visual display elements including a battery level indicator 424. The battery level indicator may be used to show the current state of the battery, that the battery is charging, that the battery is fully charged, and so on. The biosensor may include an audible alarm and indicator 426. The audible alarm may be triggered based on processing performed by the mobile device, performed in a cloud-computing environment, and so on. In embodiments, the audible alarm may be triggered by an algorithm based on risk. The audible alarm may be supplemented with visual indications. In embodiments, the biosensor may indicate an alert using LEDs.

The biosensor 410 may include a function selector 428. The function selector may be a button, a touch point, and so on. The function selector may be used to select one or more functions and/or features of the biosensor. The function selector may be used to acknowledge an alert, clear an alert, indicate a false alert, and so on. The function selector may also be used to select among various biosensor information items. The biosensor may include an information display 430. The information display may be used to indicate any appropriate information to the person using the biosensor. The information display may include various pieces of information useful to the person including a clock interface. The information display may also include an antenna connectivity readout and other useful information. The biosensor may include a programmable indicator 432. The indicator may be used for user selectable purposes or may be selected or preset for other purposes such as research purposes. The biosensor may include a level indicator 434. The level indicator may include one or more lights used to display various levels appropriate to the biosensor technique. In embodiments, the programmable indicator may include a tri-color LED ANS level indicator.

A mat 420 may be used for charging the biosensor 410. While a mat is shown, other appropriate charging techniques may be imagined including a box, a stand, and so on. Various charging techniques including inductive charging, rapid charging, trickle charging, and so on, may be used to charge the biosensor. Power to the charging mat may be provided by any appropriate technique. In embodiments, power is applied to the charging mat using a USB connection. The mat may be used for other purposes including transferring data to or from the biosensor, programming the biosensor, and so on. In embodiments, clinical data may be synchronized while the biosensor is being charged. As mentioned above, the clinical data may be synchronized using wireless or wired techniques.

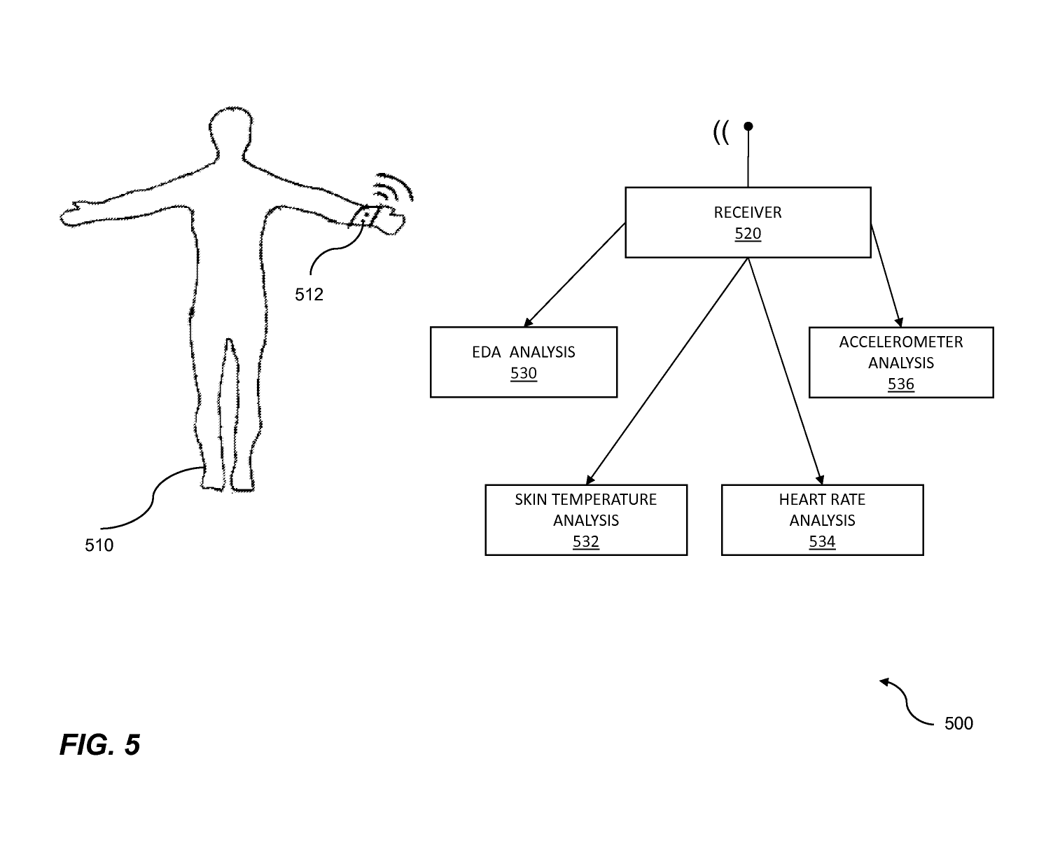

FIG. 5 is a diagram representing physiological analysis. A system 500 may analyze data collected from a person 510 as he or she interacts with a computer. The person 510 may have a biosensor 512 attached to him or her for the purpose of collecting mental state data. The biosensor 512 may be placed on the wrist, palm, hand, head, or other part of the body. In some embodiments, multiple biosensors may be placed on the body in multiple locations. In embodiments, multiple biosensors may be placed simultaneously on an individual including on a right wrist, a left wrist, a right ankle, and a left ankle The biosensor 512 may include detectors for physiological data such as electrodermal activity, skin temperature, accelerometer readings, and the like. Other detectors for physiological data may be included as well, such as heart rate, blood pressure, EKG, EEG, further brain waves, and other physiological detectors. The biosensor 512 may transmit collected information to a receiver 520 using wireless technology such as Wi-Fi, Bluetooth, 802.11, cellular, or other bands. In other embodiments, the biosensor 512 may communicate with the receiver 520 by other methods such as a wired interface or an optical interface. The receiver may provide the data to one or more components in the system 500. In some embodiments, the biosensor 512 may record multiple types of physiological information in memory for later download and analysis. In some embodiments, the download of recorded physiological data may be accomplished through a USB port or other wired or wireless connection.

Electrodermal activity may be collected in some embodiments. It may either be collected continuously, every second, four times per second, eight times per second, 32 times per second, or on some other periodic basis. Or, in some embodiments, electrodermal activity may be collected on an intermittent basis. The electrodermal activity may be recorded and stored onto a disk, a tape, flash memory, a computer system, or streamed to a server. The electrodermal activity may be analyzed 530 to indicate arousal, excitement, boredom, or other mental states based on observed changes in skin conductance. Skin temperature may be collected and/or recorded on a periodic basis. In turn, the skin temperature may be analyzed 532. Changes in skin temperature may indicate arousal, excitement, boredom, or other mental states. Heart rate may be collected and recorded, and may also be analyzed 534. A high heart rate may indicate excitement, arousal, or other mental states. Accelerometer data may be collected and used to track one, two, or three dimensions of motion. The accelerometer data may be recorded. The accelerometer data may be used to create an actigraph showing an individual's activity level over time. The accelerometer data may be analyzed 536 and may indicate a sleep pattern, a state of high activity, a state of lethargy, or other states. The various forms of data collected by the biosensor 512 may be used along with the facial data captured by the webcam in the analysis of mental state. Contextual information may be based on one or more of skin temperature and/or accelerometer data.

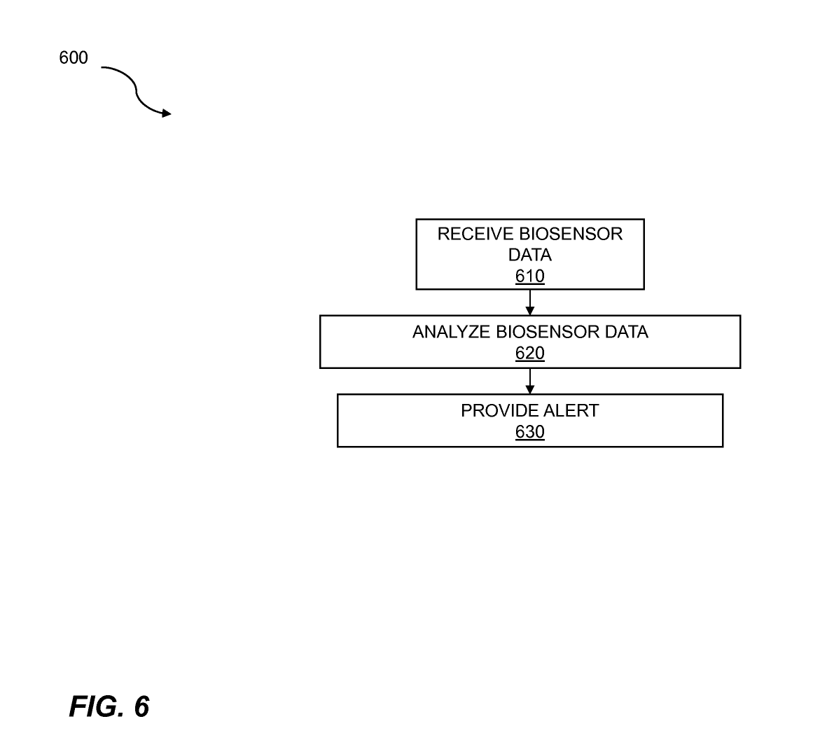

FIG. 6 is a flow diagram for providing alerts. A flow 600 describes a computer-implemented method for analyzing biosensor data and for providing alerts based on the analysis. The flow 600 may be a continuation of a previous flow or may stand on its own. The flow 600 includes receiving biosensor data 610. The biosensor data may be received from any of a variety of biosensors which may be monitoring a person. Biosensor data may be received from a caregiver. The biosensors may capture a variety of information including electrodermal activity, heart rate, heart rate variability, skin temperature, and so on. Accelerometer data may also be captured. A person may wear the biosensor or biosensors, or the sensors may be attached to a person, placed adjacent to a person, and so on. Communication with the biosensors may be made via wired or wireless techniques. Biosensor data is received from the person 610. A mobile device such as a cell phone, PDA, tablet, laptop, or other mobile device may capture the biosensor data. In embodiments, the mobile device may be incorporated as part of one or more biosensors. The biosensor data may be received via a wired link, a wireless link, or other appropriate communications mode. The biosensor data may be encoded prior to transmission for analysis. The flow 600 may include receiving captured biosensor data 610 from a person into a web-server computer system.

The flow 600 continues with analyzing biosensor data 620 which was captured from the person. The analyzing of the biosensor data may include processing, where the processing is performed, in embodiments, in a cloud-computing environment. The analyzing of biosensor data may be performed in any computational server environment. In other embodiments, a mobile device such as a cell phone, PDA, tablet, laptop, or other mobile device performs the processing. The analyzing may be based on analysis of biosensor data from an individual person or from a plurality of people. The analyzing may include identifying atypical brain activity. The atypical brain activity to be identified may include a SUDEP risk, an epileptiform seizure, a drug reaction, stress, depression, compulsive behavior, ictal brain activity, inter-ictal brain activity, tonic-clonic seizures, partial complex seizures, pain, anxiety, panic attack, epileptic seizures, sleep disorder, respiratory sleep problems, heart attack, depression, stress, reaction to medication, bipolar attack, distracted driving, concussion, stroke, autistic reaction, ADHD behavior, boredom, being startled, being in a mood, eczema outbreak, fragile X syndrome reaction, obsessive-compulsive disorder, phobia, post-traumatic stress disorder, and social anxiety disorder. The analysis may include identifying a threshold value or a plurality of threshold values. The threshold value or plurality of threshold values identified may be based on the biosensor data of an individual person or on a plurality of people.

The flow 600 continues with providing an alert 630. An alert may be provided to the person using the one or more biosensors or to one or more other people. An alert may be based on atypical brain activity, which may be detected by the one or more biosensors and/or by the analysis. The alert or plurality of alerts may be provided by visual means and/or by audible means. The alerts may be presented using one or more of lights, sounds, alphanumeric messages, audio messages, prerecorded messages, and so on. The alert may be sent to the person or persons using the biosensors, a caregiver looking after the person, one or more relatives of the person, the person's physician, and so on. An alert may be used to further train the computer algorithm to identify any atypical brain activity of the person using the biosensors. An alert may be a false alert. In the event that the alert is a false alert, the alert sent to the person may need to be reset. Resetting of the false alert may be used to improve an algorithm used for the identifying. Various steps in the flow 600 may be changed in order, repeated, omitted, or the like without departing from the disclosed inventive concepts. Various embodiments of the flow 600 may be included in a computer program product embodied in a non-transitory computer readable medium that includes code executable by one or more processors.

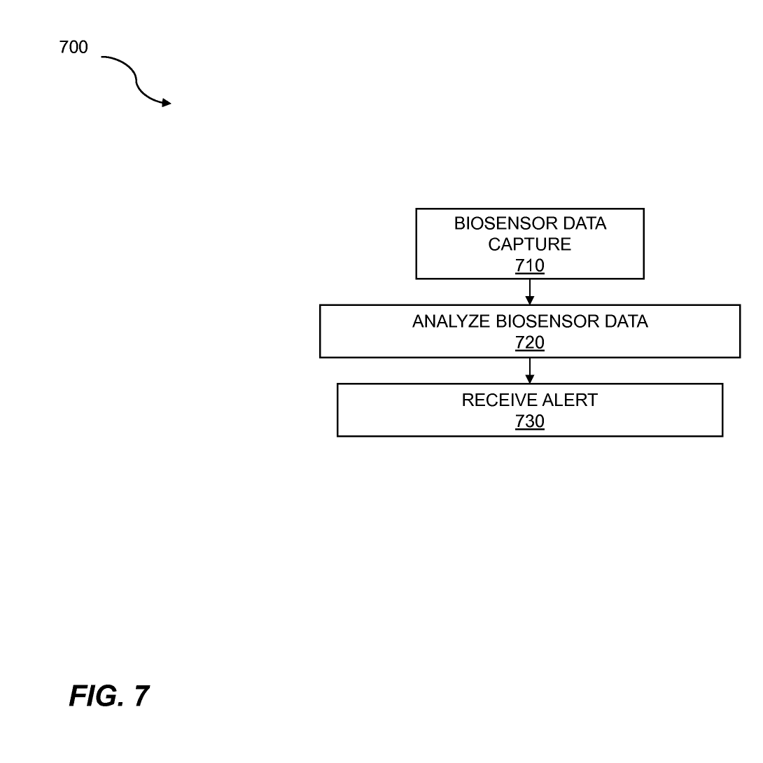

FIG. 7 is a flow diagram for receiving alerts. A flow 700 describes a computer-implemented method for analyzing biosensor data and receiving alerts based on the analysis. The flow 700 may be a continuation of a previous flow or may stand on its own. The flow 700 may include capturing biosensor data 710. The biosensor data may be captured from one or move biosensors being used by a person. The biosensor data may include one or more of electrodermal activity, heart rate, heart rate variability, skin temperature, and so on. In addition, data about the person may be captured from one or more accelerometers. As mentioned above, the biosensors may be worn by the person, attached to the person, adjacent to the person and so on. The biosensor data from the person may be captured into a processor for analysis. The processor may be a mobile device that is incorporated as part of the biosensor. The processor may be a web-based service, a cloud-based service, and so on. In embodiments, the processor may be a mobile device such as a cell phone, PDA, tablet, laptop, or other mobile device.

The flow 700 continues with analyzing biosensor data 720 captured from the person. The analyzing may include identifying atypical brain activity. The atypical brain activity may include a SUDEP risk, a epileptiform seizure, a drug reaction, stress, depression, compulsive behavior, ictal brain activity, inter-ictal brain activity, tonic-clonic seizures, partial complex seizures, pain, anxiety, panic attack, epileptic seizures, sleep disorders, respiratory sleep problems, heart attack, depression, stress, reaction to medication, bipolar attack, distracted driving, concussion, stroke, autistic reaction, ADHD behavior, boredom, being startled, being in a mood, eczema outbreak, fragile X syndrome reaction, obsessive-compulsive disorder, phobia, post-traumatic stress disorder and social anxiety disorder. The analyzing may be based on a threshold, where the threshold may be determined for an individual person or based on biosensor data from a plurality of people. Analysis may be performed on a web-server computer system, on a cloud-based service, and so on.

The flow 700 continues with receiving an alert 730. An alert may be based on biosensor data which was captured from the person using one or more biosensors, where the biosensors obtain data for electrodermal activity, heart rate, heart rate variability, skin temperature, and so on. An alert or plurality of alerts may be received by the biosensor and displayed to the person wearing the biosensor. The alert or plurality of alerts may be received using any of a variety of appropriate techniques including wired techniques, wireless techniques, and so on. An alert may be received from a caregiver. The alert may be received based on analysis which was performed on a web service, a cloud-based service, a mobile device, a portable device, or another appropriate device. The web service or other service may analyze the biosensor data to identify atypical brain activity. The analysis may be based on a threshold value. The threshold value may be based on biosensor data captured from the person using the biosensor, or may be determined based on biosensor data captured from a plurality of people. The alert may be received as a result of the analysis. Various steps in the flow 700 may be changed in order, repeated, omitted, or the like without departing from the disclosed inventive concepts. Various embodiments of the flow 700 may be included in a computer program product embodied in a non-transitory computer readable medium that includes code executable by one or more processors.

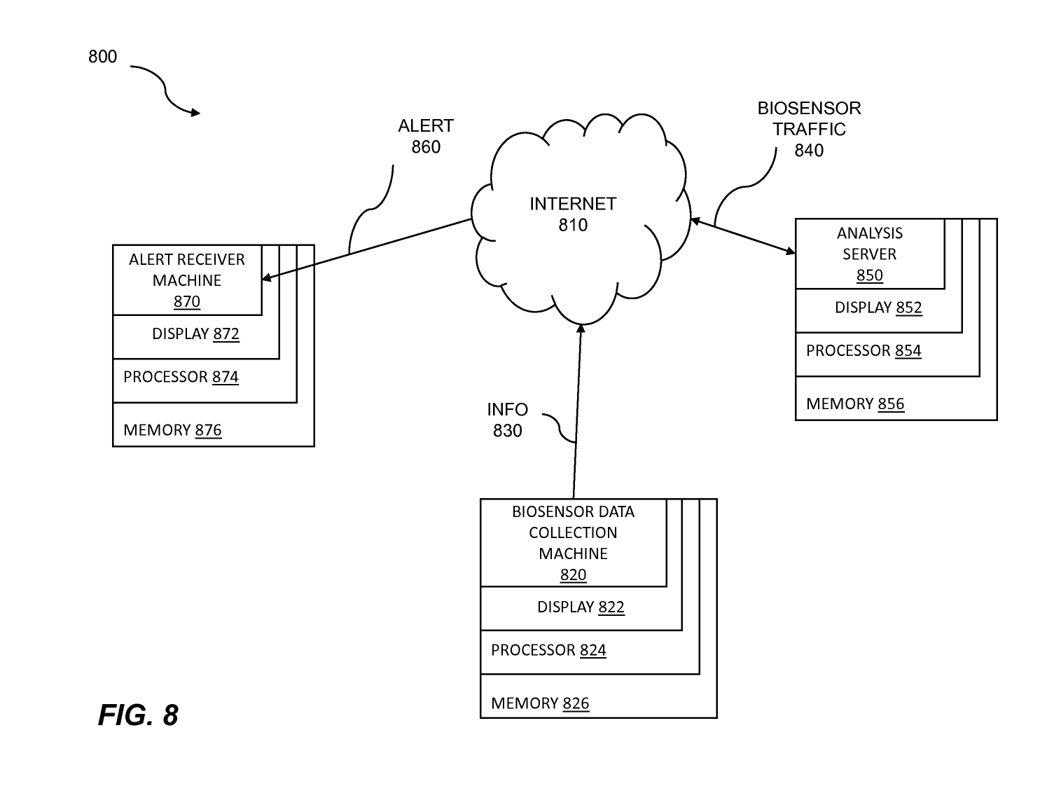

FIG. 8 is a system diagram for physiology analysis. A system 800 may include a biosensor collection machine 820, an analysis server 850, and an alert receiver machine 870. The biosensor data collection machine 820, the analysis server 850, and the alert receiver machine 870 may communicate over the Internet 810 or another computer network. The biosensor collection machine 820 has a memory 826 which stores instructions, and one or more processors 824 attached to the memory 826 wherein the one or more processors 824 can execute instructions stored in the memory 826. The memory 826 may be used for storing instructions, for storing mental state data, for system support, and the like. The biosensor data collection machine 820 also can have an Internet connection to carry viewer mental state information 830, and a display 822 that may present various information. The display may be any electronic display, including but not limited to, a computer display, a laptop screen, a net-book screen, a tablet computer screen, a cell phone display, a mobile device display, a remote with a display, a television, a projector, or the like. The collection machine 820 may be able to collect biosensor data from one or more people who may have a biosensor attached to them. In some embodiments there can be multiple biosensor data collection machines 820 in a system that each may collect biosensor data. The biosensor data collection machine 820 may capture electrodermal activity, skin temperature, accelerometer data and other types of biosensor data. The biosensor data, a subset of the biosensor data, or an initial analysis of the biosensor data (performed by the biosensor data collection machine 820) may be communicated as information 830 across a network and received at the analysis server 850. The information 830 may be communicated to the analysis server 850 without any intervening computation. In some embodiments, the information 830 may be manipulated and resulting biosensor information may be received by the analysis server.

The analysis server 850 may have a connection to the Internet 810 to enable mental state information 840 to be received by the analysis server 850. Further, the analysis server 850 may have a memory 856 which stores instructions, data, biosensor data, mental state data, help information and the like, and one or more processors 854 coupled to the memory 856 wherein the one or more processors 854 can execute instructions. The analysis server 850 can use its Internet, or other computer communication method, to obtain mental state information 840. The analysis server 850 can have a display 852 that may present various types of information including analysis data, analysis results, and so on. The display may be any electronic display, including but not limited to, a computer display, a laptop screen, a net-book screen, a tablet computer screen, a cell phone display, a mobile device display, a remote with a display, a television, a projector, or the like. The analysis computer 850 may receive mental state information collected from a plurality of people from the biosensor data collection machine or machines 820, and may aggregate biosensor information on the plurality of people. Analysis may be performed by the analysis server 850 (web service) through cloud computation of at least one of the autonomic data, a subset of the autonomic data, or an initial analysis of the autonomic data. The analysis server 850 may perform a computer-implemented method for physiology analysis comprising: receiving captured biosensor data from a person into a web-server computer system; analyzing the biosensor data from the person which was captured to identify atypical brain activity; and providing an alert based on the atypical brain activity that was detected.

An alert receiver machine 870 can have a memory 876 which stores instructions, and one or more processors 874 attached to the memory 876 wherein the one or more processors 874 can execute instructions stored in the memory 876. The memory 876 may be used for storing instructions, for storing mental state data, for storing biosensor data, for system support, and the like. The alert receiver machine 870 also may have an Internet connection to receive alerts 860, and a display 872 that may present the alerts along with various advertisements, products, or services to one or more viewers. The display may be any electronic display, including but not limited to, a computer display, a laptop screen, a net-book screen, a tablet computer screen, a cell phone display, a mobile device display, a remote with a display, a television, a projector, or the like.

In at least one embodiment, the biosensor collection machine 820 and the analysis server 850 functions are combined into one machine. In at least one embodiment, the biosensor collection machine 820 and the alert receiver machine 870 functions are combined into one machine. The system 800 may include code for capturing biosensor data from a person; code for sending the biosensor data to a mobile device; code for providing the biosensor data to a cloud computing environment; code for processing the biosensor data to identify atypical brain activity; and code for providing an alert based on the atypical brain activity that was detected. The system 800 may include code for capturing electrodermal activity data on an individual into a computer system wherein the electrodermal activity data provides information on physiology of the individual and wherein the electrodermal activity data is captured through a sensor; code for receiving analysis from a web service wherein the analysis is based on the electrodermal activity data on the individual which was captured; and code for rendering an output related to the physiology based on the analysis which was received.

Each of the above methods may be executed on one or more processors on one or more computer systems. Embodiments may include various forms of distributed computing, client/server computing, and cloud based computing. Further, it will be understood that for each flow chart in this disclosure, the depicted steps or boxes are provided for purposes of illustration and explanation only. The steps may be modified, omitted, or re-ordered and other steps may be added without departing from the scope of this disclosure. Further, each step may contain one or more sub-steps. While the foregoing drawings and description set forth functional aspects of the disclosed systems, no particular arrangement of software and/or hardware for implementing these functional aspects should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. Each element of the block diagrams and flowchart illustrations, as well as each respective combination of elements in the block diagrams and flowchart illustrations, illustrates a function, step or group of steps of the methods, apparatus, systems, computer program products and/or computer-implemented methods. Any and all such functions may be implemented by computer program instructions, by special-purpose hardware-based computer systems, by combinations of special purpose hardware and computer instructions, by combinations of general purpose hardware and computer instructions, by a computer system, and so on. Any and all of which may be generally referred to herein as a “circuit,” “module,” or “system.”

A programmable apparatus that executes any of the above mentioned computer program products or computer implemented methods may include one or more processors, microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are not limited to applications involving conventional computer programs or programmable apparatus that run them. It is contemplated, for example, that embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized. The computer readable medium may be a non-transitory computer readable medium for storage. A computer readable storage medium may be electronic, magnetic, optical, electromagnetic, infrared, semiconductor, or any suitable combination of the foregoing. Further computer readable storage medium examples may include an electrical connection having one or more wires, a portable computer diskette, a hard disk, a random access memory (RAM), a read-only memory (ROM), an erasable programmable read-only memory (EPROM), Flash, MRAM, FeRAM, phase change memory, an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. Each thread may spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the entity causing the step to be performed.

While the invention has been disclosed in connection with preferred embodiments shown and described in detail, various modifications and improvements thereon will become apparent to those skilled in the art. Accordingly, the spirit and scope of the present invention is not to be limited by the foregoing examples, but is to be understood in the broadest sense allowable by law.

What is claimed is:

1. A computer-implemented method for physiology analysis comprising:
capturing biosensor data from a person;
sending the biosensor data to a mobile device;
providing the biosensor data to a cloud-computing environment;
processing the biosensor data to identify atypical brain activity; and
providing an alert based on the atypical brain activity that was detected.

2. The method of claim 1 wherein the biosensor data includes one or more of electrodermal activity, accelerometer data, and skin temperature.

3. The method of claim 1 wherein the processing is performed in the cloud-computing environment.

4. The method of claim 1 wherein the mobile device is incorporated as part of a biosensor.

5. The method of claim 1 wherein the atypical brain activity comprises a SUDEP risk, an epileptiform seizure, a drug reaction, stress, depression, compulsive behavior, ictal brain activity, inter-ictal brain activity, a tonic-clonic seizure, a partial complex seizure, pain, anxiety, a panic attack, an epileptic seizure, a sleep disorder, a respiratory sleep problem, an heart attack, a reaction to medication, a bipolar attack, distracted driving, a concussion, a stroke, an autistic reaction, ADHD behavior, boredom, being startled, being in a mood, an eczema outbreak, a fragile X syndrome reaction, an obsessive-compulsive reaction, a phobia, a post-traumatic stress disorder reaction, or a social anxiety disorder.

6. The method of claim 1 further comprising training a computer algorithm for the processing the biosensor data to identify atypical brain activity.

7. The method of claim 6 wherein cancelling an alert is used to further train the computer algorithm to identify the atypical brain activity.

8. The method of claim 6 wherein the training the computer algorithm is based on feedback after the alert.

9. The method of claim 1 further comprising collecting quantitative self-report information from the person.

10. The method of claim 1 wherein the alert is sent to one or more of the person, a caregiver, a relative, a physician.

11. The method of claim 1 further comprising dosing the person based on the atypical brain activity that was identified.

12. The method of claim 1 wherein the processing the biosensor data to identify atypical brain activity is based on a threshold value.

13. A computer program product embodied in a non-transitory computer readable medium for physiology analysis, the computer program product comprising:
code for capturing biosensor data from a person;
code for sending the biosensor data to a mobile device;
code for providing the biosensor data to a cloud-computing environment;
code for processing the biosensor data to identify atypical brain activity; and
code for providing an alert based on the atypical brain activity that was detected.

14. A system for physiology analysis comprising:
a memory which stores instructions;
one or more processors coupled to the memory wherein the one or more processors, when executing the instructions which are stored, are configured to:
capture biosensor data from a person;
send the biosensor data to a mobile device;
provide the biosensor data to a cloud-computing environment;
process the biosensor data to identify atypical brain activity; and
provide an alert based on the atypical brain activity that was detected.

15-16. (canceled)
